(19) United States
(12) Patent Application Publication
(10) Pub. No.: US 2021/0372919 A1
(43) Pub. Date: Dec. 2, 2021

(54) BLACK-ICE AND STANDING-WATER DETECTION SYSTEM

(71) Applicant: Murata Manufacturing Co., Ltd., Nagaokakyo-shi, Kyoto-fu (JP)

(72) Inventors: Yuji NAKANISHI, Smyrna, GA (US); Yuichi KUSHIHI, Nagaokakyo-shi (JP)

(21) Appl. No.: 16/647,046

(22) PCT Filed: Sep. 18, 2018
(86) PCT No.: PCT/US2018/051483

§ 371 Date: Mar. 13, 2020

Related U.S. Application Data
(60) Provisional application No. 62/560,286, filed on Sep. 19, 2017.

(51) Int. Cl. G01N 21/3554; G01N 21/31; G06K 9/00; H04N 5/232; B60W 40/06; B60Q 1/00

(52) U.S. Cl. CPC G01N 21/3554; G01N 21/314; G06K 9/00798; G06K 9/00825; G01N 2033/1873; H04N 5/23296; B60W 40/06; B60Q 1/0023; H04N 5/232127

(57) ABSTRACT

A detector system for a vehicle includes a camera that provides image data, a radiation source, a radiation detector that detects reflected radiation, and a processor that determines a shape of a road based on the image data, adjusts a focus location of the radiation detector based on the shape of the road, and detects black ice and/or standing water based on a ratio of a first reflected wavelength and a second reflected wavelength of detected reflected radiation.

### BACKGROUND OF THE INVENTION

### 1. Field of the Invention

The present invention relates to black-ice and standing-water detection. More specifically, the present invention relates to a detector system that uses infrared (IR) radiation to detect black ice and standing water.

### 2. Description of the Related Art

As described in Namcheol Baik et al., “METHODS OF MONITORING THE ROAD SURFACE FREEZING SECTIONS FOR PREVENTION OF ICY WINTER ROAD DISASTERS ON THE HIGHWAYS,” Proceeding the 6th Civil Engineering Conference in Asia Region: Embracing the Future through Sustainability, 2013, pp. SS6-17-SS6-23 (Baik et al.), one known method to detect icy road conditions uses two cameras to detect ice on the road. One camera is a polarized camera, and the other camera is a normal camera. Images from the two cameras are compared. But, as admitted in Baik et al., this known method using two cameras is not accurate enough to be reliably used in a vehicle.

Another known method to detect icy road conditions is sensing a vehicle's tire friction. If a change in tire friction is sensed, then this data is uploaded to a cloud map and distributed to other vehicles. But this known method is dangerous for the occupants of the first vehicle that first encounter the ice on the road.

JP Application No. 2009-042115 A discloses a road-condition detector that uses a camera to detect moisture on the road 20 m to 60 m in front of the vehicle based on, in part, the detection of moisture 1 m to 5 m in front of a vehicle from a near-IR sensor. But this known IR road-condition detector does not account for the shape or characteristics of the road. For example, the known IR road-condition detector is not able to account for a curve in the road. Not detecting changes or characteristics in the road can lead to false detections of moisture not on the road and lead to not detecting moisture on a curved road until it is too late to slow down. The road-condition detector of JP Application No. 2009-042115 A uses near-site brightness to eliminate influence from environmental brightness.

### SUMMARY OF THE INVENTION

To overcome the problems described above, preferred embodiments of the present invention provide IR black-ice and standing-water detector systems that use a camera and either a radiation detector or a radiation detector array to detect black ice and standing water. The camera can be used to detect a road's shape, to adjust the direction of the radiation detector based on the road's shape, and to detect the presence of other vehicles.

According to a preferred embodiment of the present invention, a detector system for a vehicle includes a camera that provides image data, a radiation source, a radiation detector that detects reflected radiation, and a processor that determines a shape of a road based on the image data, adjusts a focus location of the radiation detector based on the shape of the road, and detects black ice and/or standing water based on a ratio of a first reflected wavelength and a second reflected wavelength of detected reflected radiation.

The radiation source preferably emits radiation in a first IR band and a second IR band. Preferably, the first IR band includes IR radiation having wavelengths below about 2.15 μm, and the second IR band includes IR radiation having wavelengths above about 2.15 μm. The radiation source preferably emits radiation with a first emitted wavelength of about 2.10 μm and a second emitted wavelength of about 2.25 μm. The radiation source preferably emits radiation having wavelengths of between about 300 nm and about 8 μm.

Preferably, black ice is detected if the ratio of the first reflected wavelength and the second reflected wavelength is about 0.40, and standing water is detected if the ratio of the first reflected wavelength and the second reflected wavelength is about 0.85.

Preferably the processor detects another vehicle on the road based on the image data and adjusts the focus location of the radiation detector based on the another vehicle. The processor detects lanes in the road based on the image data and adjusts the focus location of the radiation detector based on the lanes in the road.

The detector system further preferably includes a zoom lens and a gear motor, wherein the zoom lens changes a focus of the radiation detector and the gear motor changes a direction of the radiation detector. Preferably, the radiation detector includes a lens that focuses the detected reflected radiation on a splitter, a first filter that receives a first portion of split radiation from the splitter and that outputs radiation having the first reflected wavelength, a first photodiode that receives the radiation having the first reflected wavelength, a second filter that receives a second portion of split radiation from the splitter and that outputs radiation having the second reflected wavelength, and a second photodiode that receives the radiation having the second reflected wavelength.

According to a preferred embodiment of the present invention, a detector system for a vehicle includes a camera that provides image data, a radiation source, an array of radiation detectors that detects reflected radiation, and a processor that determines a shape of a road based on the image data, selects a radiation detector in the array of radiation detectors based on the shape of the road, and detects black ice and/or standing water based on a ratio of a first reflected wavelength and a second reflected wavelength of detected reflected radiation by the selected radiation detector.

The radiation source preferably emits radiation in a first IR band and a second IR band. Preferably, the first IR band includes IR radiation having wavelengths below about 2.15 μm, and the second IR band includes IR radiation having wavelengths above about 2.15 μm. The radiation source preferably emits radiation with a first emitted wavelength of about 2.10 μm and a second emitted wavelength of about 2.25 μm. The radiation source preferably emits radiation having wavelengths of between about 300 nm and about 8 μm. Preferably, black ice is detected if the ratio of the first reflected wavelength and the second reflected wavelength is about 0.40, and standing water is detected if the ratio of the first reflected wavelength and the second reflected wavelength is about 0.85.

Preferably, the processor detects another vehicle on the road based on the image data and adjusts the selection of the radiation detector in the array of radiation detectors based on the another vehicle. Preferably, the processor detects lanes in the road based on the image data and adjusts the selection of the radiation detector in the array of radiation detectors based on the lanes in the road.

According to a preferred embodiment of the present invention, a vehicle including the detector system of one of the various preferred embodiments of the present invention. On a straight road, the radiation detector is preferably focused about 60 m in front of the vehicle. The detector system preferably automatically applies brakes of the vehicle if black ice or standing water is detected. The radiation source preferably is a headlight of the vehicle. The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

FIG. 1 shows a vehicle with a detector according to a preferred embodiment of the present invention on a straight road.

FIG. 2 shows vehicle with the detector of FIG. 1 on a curved road.

FIG. 3 shows a schematic diagram of a radiation detector.

FIG. 4 shows a schematic diagram of a radiation source.

FIG. 5 shows a detector system with the radiation detector shown in FIG. 3 and the radiation source shown in FIG. 4.

FIG. 6 an example of a device that includes the radiation detector shown in FIG. 3.

FIG. 7 shows a mounting location of the device shown in FIG. 6.

FIGS. 8 and 9 show a radiation detector array.

FIG. 10 shows a detector system with the radiation detector shown in FIG. 8 and the radiation source shown in FIG. 4.

FIGS. 11-14 show images with different road configurations.

### DETAILED DESCRIPTION OF PREFERRED EMBODIMENTS

Detector systems according to preferred embodiments of the present invention detect black-ice and standing-water conditions on roads by using reflection of IR radiation. Black ice is a thin coating of ice on a road. While the ice is not black, the road visible through the thin coating of ice is. Standing water can create hydroplaning conditions. For example, a vehicle travelling at 50 mph can hydroplane in 3 mm of standing water.

It is important to detect ice or water in sufficient time to allow a vehicle's driver to slow the vehicle down before the driving over or through the ice or water on the road. For a vehicle travelling at 70 mph on a dry road to slow down to 20 mph before driving over ice or water on the road, the detector system should detect the ice or water at least 60 m ahead of the vehicle. Although this 60 m distance allows for sufficient time for a typical vehicle to slow down, other distances can also be used. For example, distances can be adjusted based on the vehicle's speed, and longer distances can be used for vehicles, such as trucks or tractor trailers, that take longer to slow down and for slippery road conditions such as wet or snow-covered roads (but vehicles should not be travelling at 70 mph on roads with slippery road conditions). On a curvy road, 60 m ahead may not be the road, but could be a building, field, etc., in which case 60 m ahead cannot be used to detect ice or water.

The detector systems according to preferred embodiments of the present invention include a radiation source and a radiation detector. The radiation source can be a vehicle's headlamp, including, for example, a halogen or xenon lamp. It is also possible to use other suitable radiation sources. The radiation detector can be an IR radiation detector such as an InGaAs or PbS photodetector. It is also possible to use other suitable radiation detectors. IR radiation is emitted from the radiation source, and the radiation detector detects the IR radiation reflected from the road, and any ice or water on the road. The detector system can use two different wavelengths of IR radiation to detect black ice and standing water on a road. The detector system can use, for example, the IR detection techniques disclosed in U.S. Patent Application Publication Nos. 2015/0120092 and 2015/0120093.

U.S. Patent Application Publication Nos. 2015/0120092 and 2015/0120093 disclose detecting black ice using two IR wavelengths. Although U.S. Patent Application Publication Nos. 2015/0120092 and 2015/0120093 disclose techniques that detect black ice and standing water more accurately than other known techniques, U.S. Patent Application Publication Nos. 2015/0120092 and 2015/0120093 do not address the location of the black ice or standing water. For example, U.S. Patent Application Publication Nos. 2015/0120092 and 2015/0120093 do not consider the shape of the road and do not verify that the reflected IR signal indicating the detection of black ice or water is from the lane that the vehicle is in and not from another lane, off of a curvy road (e.g., field, building etc.), from another vehicle, etc.

The radiation source can emit IR radiation. For example, a halogen lamp can be used as the radiation source and emits radiation, for example, between about 300 nm and about 8 p.m. To detect both ice and water, the radiation source can emit radiation in at least two different IR bands, for example, one with wavelengths below about 2.15 μm and one with wavelengths above about 2.15 μm. For example, the radiation source can emit IR radiation with a wavelength of about 2.10 μm and with a wavelength of about 2.25 μm. The radiation source can emit radiation with other wavelengths, for example, if the headlamp is used as the radiation source. At a wavelength of about 2.15 μm, the absorption characteristics of liquid water invert relative to the absorption characteristics of ice. The ratio of the radiance or reflectance of the IR radiation in the two bands can be used to determine the presence of ice or water. Depending on the selected wavelength, the ratio of the two reflected IR wavelengths can be used to determine if the reflected radiation is from ice or water. For example, if the ratio of the reflected radiation at 2.10 μm to the reflected radiation at 2.25 μm is around 0.40, then ice reflected the radiation. If the ratio of the reflected radiation at 2.10 μm to the reflected radiation at 2.25 μm is around 0.85, then water reflected the radiation.

The detected radiation at the two different wavelengths have the same or substantially the same environmental brightness within detecting tolerances. Comparing the two detected wavelengths cancels out or significantly reduces the environmental brightness, which allows the detector systems of the preferred embodiments of the present invention to not be affected by environmental brightness.

A detector system according to a preferred embodiment of the present invention can include a camera that detects the road's shape and the presence of other vehicles. The camera can be any suitable forward-looking automotive camera, including, for example, a complementary metal-oxide semiconductor (CMOS) camera. Detecting the road's shape also includes detecting the road's lanes. By determining the road's shape and the road's lanes, the radiation detector can be oriented to an area in the vehicle's lane and not to an area off the road, at a building or other structure, at another vehicle, etc. If the radiation detector is not oriented to monitor the vehicle's lane, for example, if another vehicle, building, field, etc. is detected, then the radiation detector's focus is adjusted to an area in the vehicle's lane. If another vehicle is detected in the vehicle's lane, then the radiation detector is oriented to an area just behind the detected vehicle.

The camera is preferably connected to a high-performance processor that receives image data from the camera. The high-performance processor analyzes the images data to determine the shape of the road and the presence of other vehicles on the road. Detecting or determining the shape of the road allows the radiation detector to be oriented to monitor the road, and not to be oriented to monitor off-road areas.

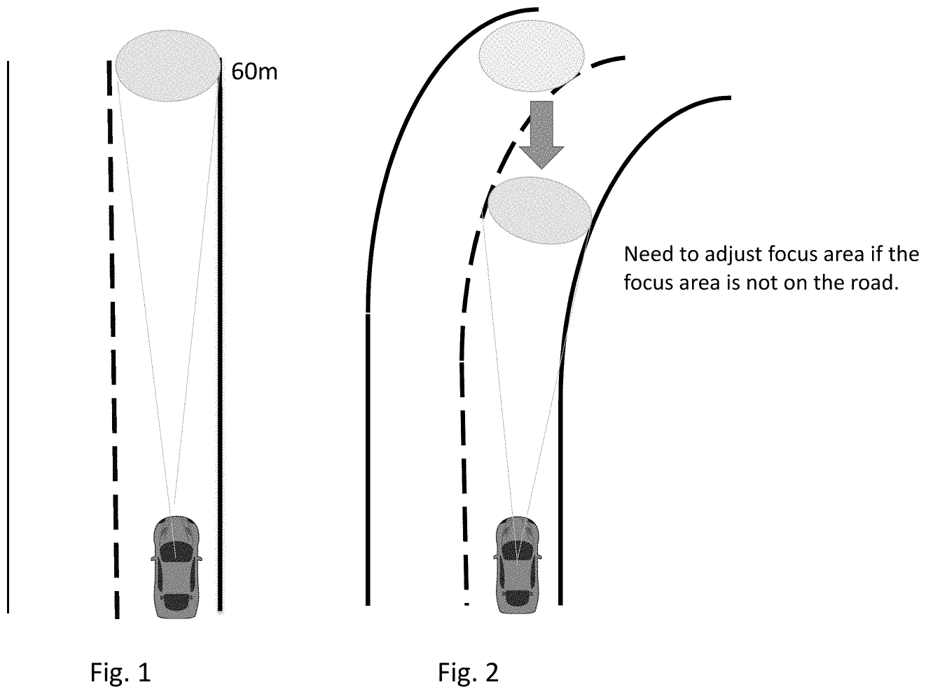

Based on the shape of the road, the radiation detector's focus distance and/or direction can be changed. For example, the radiation detector's focus can be changed using a zoom lens, and the radiation detector's direction can be changed using a motor with gears. In FIG. 1, the radiation detector is directed straight ahead with a focus distance of 60 m, and in FIG. 2, based on the curve in the road, the detector is tilted to the right and the focus distance is shortened based.

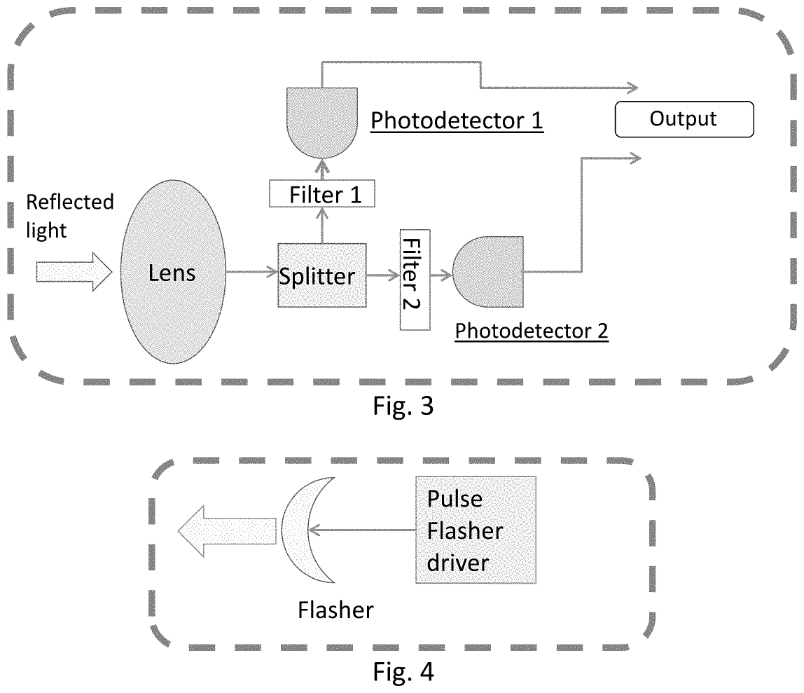

FIG. 4 shows an example of a radiation source, and FIG. 3 shows an example of radiation detector. The radiation source can include a flasher and an optional pulse flasher driver. For example, the flasher can be the vehicle's headlamp or can be separate from the vehicle's headlamp. For example, if the headlamp is an LED headlamp, then an additional halogen flasher can be used. If the headlamp is not the flasher and a separate flasher is used, then the pulse flasher driver can be used to turn the flasher on and off, which can save energy.

The radiation detector in FIG. 3 is an IR radiation detector and is tiltable. The radiation detector detects radiation emitted by the flasher that is reflected off various surfaces. The radiation detector includes a lens that focuses the reflected radiation on a splitter. The split radiation is filtered by filters 1 and 2. For example, filter 1 can filter radiation at 2.10 μm, and filter 2 can filter radiation at 2.25 μm. The radiation filtered by filter 1 is received by photodetector 1, and the radiation filtered by filter 2 is received by photodetector 2. The photodetectors 1 and 2 then output the amount of detected radiation at, for example, 2.10 μm and 2.25 μm. As explained above, the ratio of the amount of detected radiation by photodetector 1 to the amount of detected radiation by photodetector 2 can be used to detect ice or water. Because the radiation detector is oriented at an area in the vehicle's lane, then the ice or water can be detected before the vehicle encounters the ice or water.

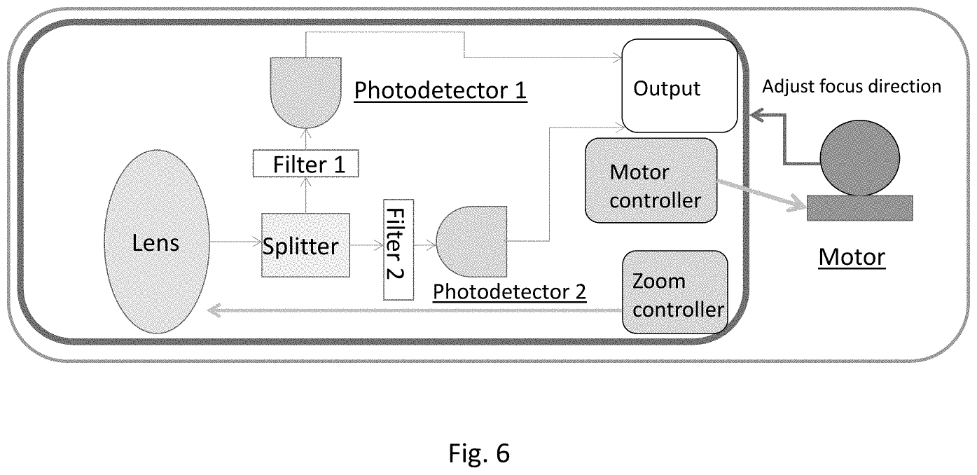

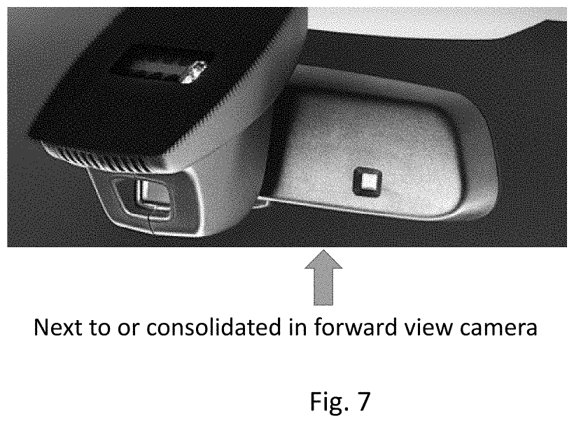

FIG. 6 shows a device that includes the radiation detector shown in FIG. 3 and a motor. In FIG. 6, the radiation detector also includes a motor controller and a zoom controller. The motor controller controls the motor included in the device. The motor adjusts the radiation detector's direction. The zoom controller adjusts the focus of the lens included in the radiation detector. Although not shown in FIG. 6, the device can also include an angular sensor that determines the orientation of the radiation detector. The device shown in FIG. 6 can be mounted next to or made integral with a vehicle's rear-view mirror, as shown in FIG. 7. As with the camera, it is possible to mount the device in other locations, including, for example, in the vehicle's grill, so long as the radiation detector can receive reflected radiation emitted by the flasher.

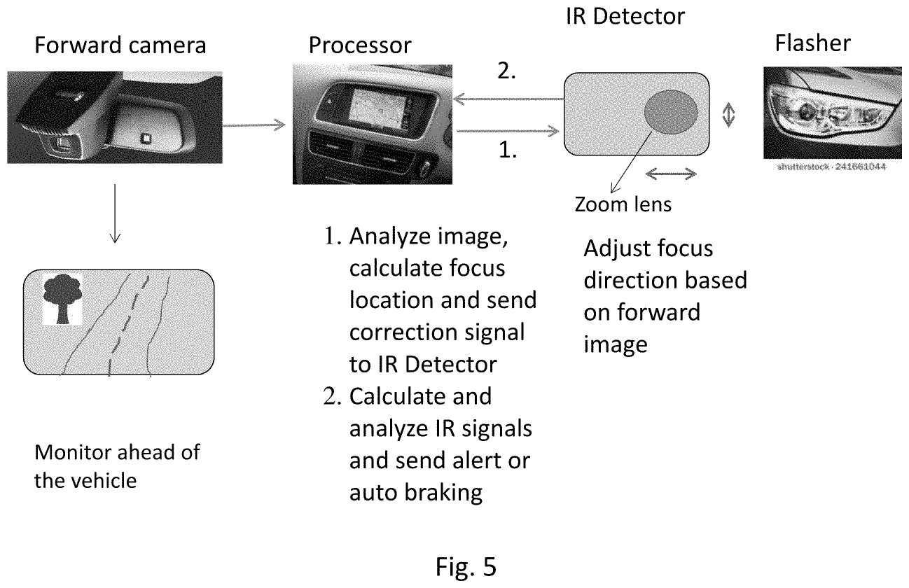

FIG. 5 shows an example of a detector system according to a preferred embodiment of the present invention. The detector system includes a camera, a processor, an IR detector, and a flasher.

In FIG. 5, the camera is included with the rear-view mirror of a vehicle, but the camera can be located in any location on the vehicle so long as the camera faces forward. The camera monitors the area in front the vehicle, including the road in front of the vehicle. The camera sends image data to a processor.

In FIG. 5, the processor is included in the vehicle's center console, e.g. in the navigation or infotainment system, but the processor can be located separate from the center console. Any suitable processor or any number of processors can be used. The processor is connected to the camera and to the IR detector. The processor analyzes the image data from the camera to determine what the IR detector's focus and orientation should be. As explained above, the IR detector should be oriented to receive reflected IR radiation from the road in front of the vehicle. If the processor determines that the road includes more than one lane, then the IR detector should be oriented to monitor the lane that the vehicle is in.

The processor also analyzes reflected radiation data from the IR detector to determine the presence of ice or water on the road. Based on the determination of the presence of ice or water, the processor can provide an alert or can provide automatic braking.

The flasher emits IR radiation, and the IR detector detects reflected IR radiation. In FIG. 5, the flasher is included in the vehicle's headlamp, but any suitable radiation source and location could be used.

Instead of tilting the IR detector, it is also possible use multiple IR detectors arranged in an array. Although using multiple IR detectors may not cover the same area as a tilting IR detector, several IR detectors can be suitable in certain applications. Arrays with a larger number of IR detectors can provide better resolution than arrays with a smaller number of IR detectors.

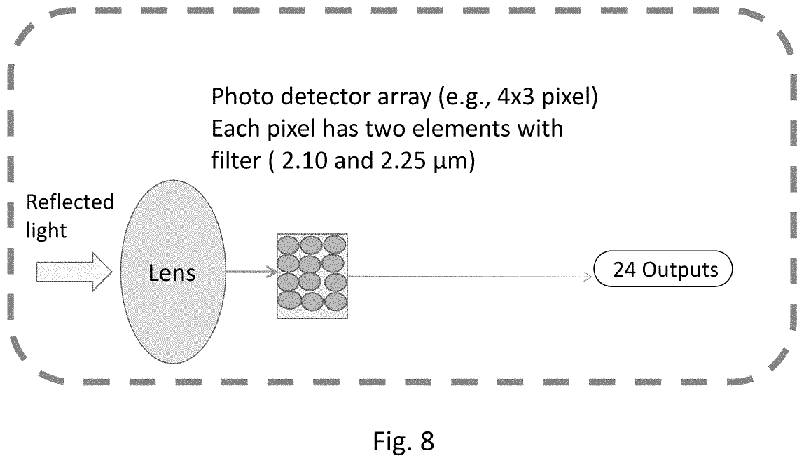

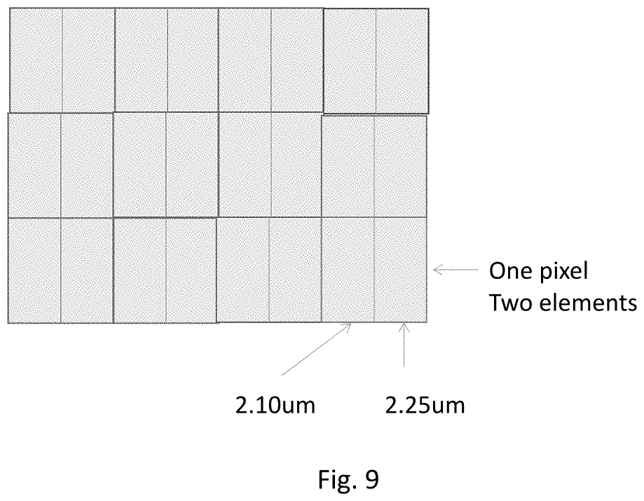

FIG. 8 shows a lens focusing reflected radiation on a radiation detector array. FIG. 9 shows a close-up of the radiation detector array. The radiation detector array is fixed and does not need a camera to determine how the radiation detector array should be oriented, although a camera can still be used to determine the road's shape as explained below. As with the radiation detector shown in FIG. 3, the radiation detector array of FIGS. 8 and 9 can be used with the radiation source shown in FIG. 4. The radiation detector array shown in FIG. 8 uses an array of radiation detectors to detect reflected radiation. FIG. 8 shows an array of 4×3 radiation detectors, but any number or arrangement of radiation detectors could be used. As shown in FIG. 9, each radiation detector (or pixel) in the array can determine the amount of radiation at, for example, 2.10 μm and 2.25 μm, which as explained above can be used to determine the presence of ice and water. In FIGS. 8 and 9, the 3×4 array would provide a total of 24 outputs, with each of the 12 radiation detectors providing two outputs. A different number of radiation detectors would provide a different number of outputs.

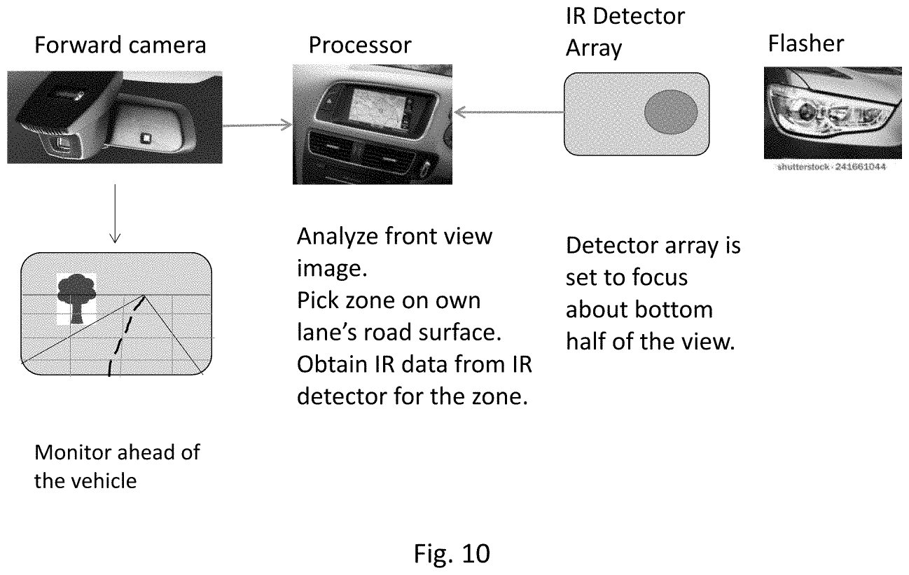

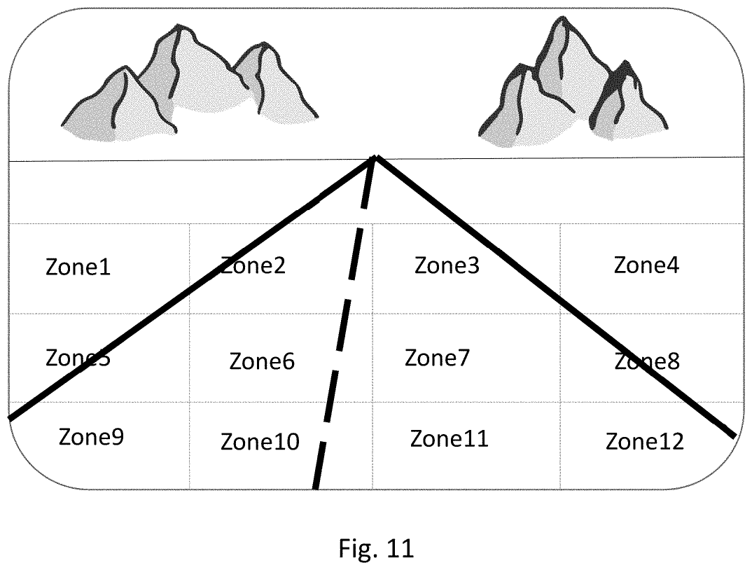

FIG. 10 shows a detector system that uses the radiation detector array shown in FIG. 8. The detector system in FIG. 10 is similar to the detector system shown in FIG. 5 in that the detector system includes a camera, processor, and flasher. In FIG. 10, a forward-looking image is divided into an array of zones, further examples of which are shown in FIGS. 11-14. Each zone corresponds to one of the radiation detectors in the radiation detector array. As shown in FIG. 10, only the lower portion of the image is divided into zones based on the assumption that the road will not be located in the upper portion of the image. The processor analyzes the image to determine in which zone or zones the road is located and which zone or zones need to be monitored for ice and water. For example, the processor can analyze the image to determine which zone includes an area 60 m in front of the vehicle in the vehicle's lane.

FIGS. 11-14 show images with different road configurations. In FIG. 11, the road extends straight ahead, and zone 3 is the best zone to monitor for ice and water because zone 3 includes an area about 60 m in front of the vehicle in the vehicle's lane. Although zones 7, 8, 11, and 12 are in the vehicle's lane, zones 7, 8, 11, and 12 are too close to the vehicle. Zones 2, 5, 6, 9, and 10 are in another lane, and zones 1 and 4 are off the road. The processor can monitor the reflected radiation from the radiation detectors in zone 3 to determine the presence of ice and water.

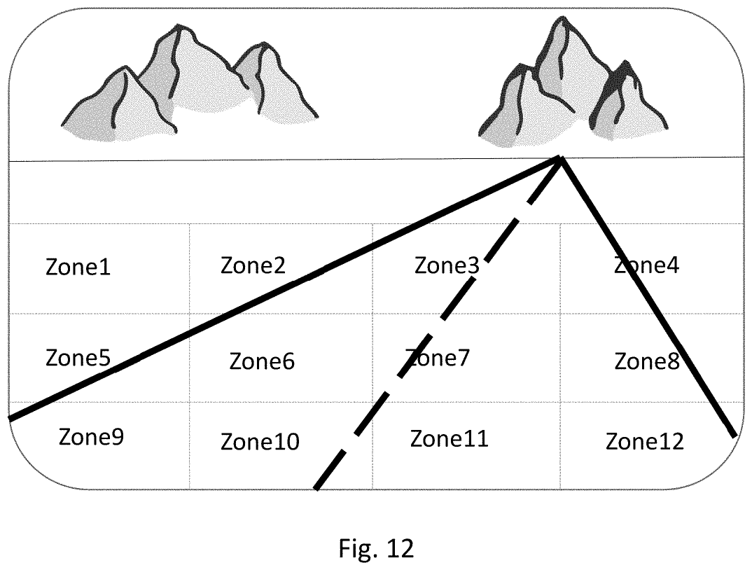

In FIG. 12, the road extends, i.e., curves, to the right, and zones 3 and 4 are almost equal in that they include an area about 60 m in front of the vehicle in the vehicle's lane, so both zones 3 and 4 can be monitored. Alternatively, only one of zones 3 and 4 be monitored. Zones 7 and 8 are almost equal but are too close to the vehicle. The other zones are too close, in the other lane, or off road. The processor can monitor the reflected radiation from the radiation detectors in zones 3 and/or 4 to determine the presence of ice and water.

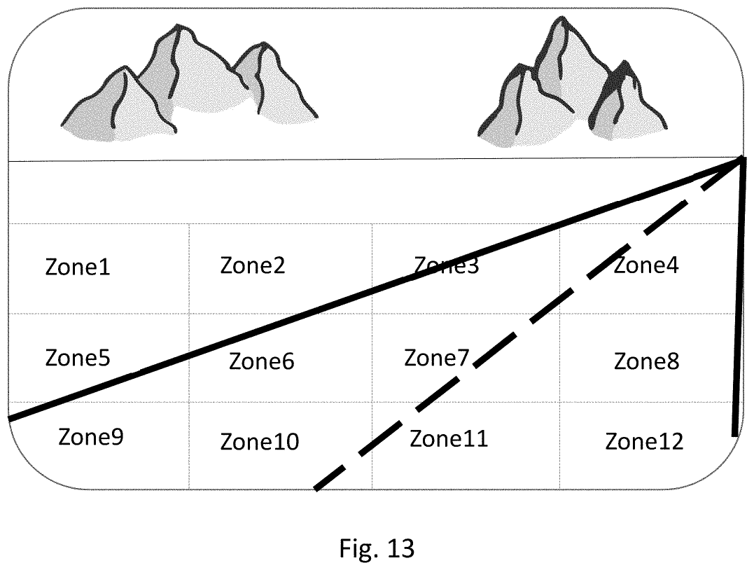

In FIG. 13, the road extends to the right even more than as shown in FIG. 12, and zone 4 is the best zone to monitor. The other zones are too close, in the other lane, or off road. The processor can monitor the reflected radiation from the radiation detectors in zone 4 to determine the presence of ice and water.

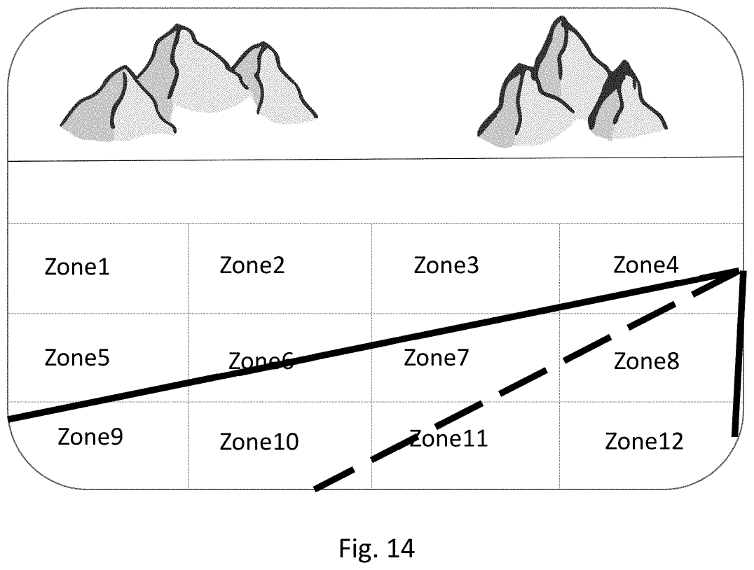

In FIG. 14, the road extends to the right even more than as shown in FIGS. 12 and 13, and zone 8 is the best zone to monitor. The other zones are too close, in the other lane, or off road. The processor can monitor the reflected radiation from the radiation detectors in zone 8 to determine the presence of ice and water.

If a larger radiation detector array is used, then the image can be divided into more zones, which improves the accuracy and reliability of the ice and water detection.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

1: A detector system for a vehicle comprising:
a camera that provides image data;
a radiation source;
a radiation detector that detects reflected radiation; and
a processor that:
determines a shape of a road based on the image data;
adjusts a focus location of the radiation detector based on the shape of the road; and
detects black ice and/or standing water based on a ratio of a first reflected wavelength and a second reflected wavelength of detected reflected radiation.

2: The detector system of claim 1, wherein the radiation source emits radiation in a first IR band and a second IR band.

3: The detector system of claim 2, wherein the first IR band includes IR radiation having wavelengths below about 2.15 μm and the second IR band includes IR radiation having wavelengths above about 2.15 μm.

4: The detector system of claim 1, wherein the radiation source emits radiation with a first emitted wavelength of about 2.10 μm and a second emitted wavelength of about 2.25 μm.

5: The detector system of claim 1, wherein the radiation source emits radiation having wavelengths of between about 300 nm and about 8 μm.

6: The detector system of claim 1, wherein black ice is detected if the ratio of the first reflected wavelength and the second reflected wavelength is about 0.40, and standing water is detected if the ratio of the first reflected wavelength and the second reflected wavelength is about 0.85.

7: The detector system of claim 1, wherein the processor:
detects another vehicle on the road based on the image data; and
adjusts the focus location of the radiation detector based on the another vehicle.

8: The detector system of claim 1, wherein the processor:
detects lanes in the road based on the image data; and
adjusts the focus location of the radiation detector based on the lanes in the road.

9: The detector system of claim 1, further comprising a zoom lens and a gear motor; wherein
the zoom lens changes a focus of the radiation detector; and
the gear motor changes a direction of the radiation detector.

10: The detector system of claim 1, wherein the radiation detector includes:
a lens that focuses the detected reflected radiation on a splitter;
a first filter that receives a first portion of split radiation from the splitter and that outputs radiation having the first reflected wavelength;
a first photodiode that receives the radiation having the first reflected wavelength;
a second filter that receives a second portion of split radiation from the splitter and that outputs radiation having the second reflected wavelength; and
a second photodiode that receives the radiation having the second reflected wavelength.

11: A detector system for a vehicle comprising:
a camera that provides image data;
a radiation source;
an array of radiation detectors that detects reflected radiation; and
a processor that:
determines a shape of a road based on the image data;
selects a radiation detector in the array of radiation detectors based on the shape of the road; and
detects black ice and/or standing water based on a ratio of a first reflected wavelength and a second reflected wavelength of detected reflected radiation by the selected radiation detector.

12: The detector system of claim 11, wherein the radiation source emits radiation in a first IR band and a second IR band.

13: The detector system of claim 12, wherein the first IR band includes IR radiation having wavelengths below about 2.15 μm and the second IR band includes IR radiation having wavelengths above about 2.15 μm.

14: The detector system of claim 11, wherein the radiation source emits radiation with a first emitted wavelength of about 2.10 μm and a second emitted wavelength of about 2.25 μm.

15: The detector system of claim 11, wherein the radiation source emits radiation having wavelengths of between about 300 nm and about 8 μm.

16: The detector system of claim 11, wherein black ice is detected if the ratio of the first reflected wavelength and the second reflected wavelength is about 0.40, and standing water is detected if the ratio of the first reflected wavelength and the second reflected wavelength is about 0.85.

17: The detector system of claim 11, wherein the processor:
detects another vehicle on the road based on the image data; and
adjusts the selection of the radiation detector in the array of radiation detectors based on the another vehicle.

18: The detector system of claim 1, wherein the processor:
detects lanes in the road based on the image data; and
adjusts the selection of the radiation detector in the array of radiation detectors based on the lanes in the road.

19: A vehicle including the detector system of claim 1.

20: The vehicle of claim 19, wherein, on a straight road, the radiation detector is focused about 60 m in front of the vehicle.

21: The vehicle of claim 19, wherein the detector system automatically applies brakes of the vehicle if black ice or standing water is detected.

22: The vehicle of claim 19, wherein the radiation source is a headlight of the vehicle.
